(19) United States
(12) Patent Application Publication
(10) Pub. No.: US 2013/0217088 A1
(43) Pub. Date: Aug. 22, 2013

(54) Method to enhance microbial gas production from unconventional reservoirs and kerogen deposits

(71) Applicant: Marcus G. Theodore, Salt Lake City, UT (US)

(72) Inventor: Marcus G. Theodore, Salt Lake City, UT (US)

(73) Assignee: Earth Renaissance Technolgies, LLC, Salt Lake City, UT (US)

(21) Appl. No.: 13/385,443

(22) Filed: Feb. 21, 2012

(51) Int. Cl. C12P 5/02

(52) U.S. Cl. 435/167

(57) ABSTRACT

A biostimulation method of the production of methane and petroleum from microbial metabolism at the margins of a basin where the organic matter is less mature and hydrologic flow systems are active.

### BACKGROUND OF THE INVENTION

1. Field

This invention relates to methane and petroleum production. More particularly, it relates to the production of methane and petroleum via biostimulation of microbial metabolism from the margins of a basin where the organic matter is less mature and hydrologic flow systems are active.

2. State of the Art

Unconventional gas deposits, such as those produced from coal beds and shales containing kerogen are new sources of methane gas. Black shales and coal beds contain carbon deposits where microbial methanogenis and modification of thermogenic gas is present at the shallower margins of a basin where the organic matter is less mature and hydrologic flows are present. These deposits contain kerogen, which is a mixture of organic chemical compounds that make up a portion of the organic matter in sedimentary rocks. It is insoluble in normal organic solvents because of the huge molecular weight (upwards of 1,000 Daltons) of its component compounds. The soluble portion is known as bitumen. Production of oil and gas from kerogen is usually accomplished under geophysical pressure and temperature conditions at deeper depths (thermogenic gas play), over long periods of time where organic material experiences more thermal cracking. When heated to the right temperatures in the Earth's crust, some types of kerogen release crude oil or natural gas. For example, oils are formed around 60-160° C. and gas is formed around 150-200° C., depending on how quickly the source rock is heated.

Kerogen is formed from the decomposition and degradation of living matter, such as diatoms, planktons, spores and pollens. In the break-down process, large biopolymers from proteins and carbohydrates begin to partially or completely dismantle. Under pressure, these dismantled components can form new geopolymers, which are the precursors of kerogen.

The formation of geopolymers account for the large molecular weights and diverse chemical compositions associated with kerogen. The smallest geopolymers are the fulvic acids, the medium geopolymers are the humic, and the largest geopolymers are the humins. When organic matter is contemporaneously deposited with geologic material, subsequent sedimentation and progressive burial or overburden provides sufficient geothermal pressures over geologic time to become kerogen. Changes such as the loss of hydrogen, oxygen, nitrogen, and sulfur and other functional groups result in isomerization and aromatization at increasing depths or burial eventually producing petroleum or methane gases.

This geophysical production of petroleum and methane gas from black shale and coal bed deposits containing kerogen is extremely slow. Consequently, new sources of natural gas require enhancing microbial gas from unconventional reservoirs. The present method described below expedites the production of petroleum and methane from unconventional gas play. It biostimulates certain bacteria and micro-organisms with sulfurous acid delivered nutrients to break down kerogen and other organic matter into petroleum and methane.

### SUMMARY OF THE INVENTION

Natural alteration of organic matter into methane by microorganisms in oxygen-depleted subsurface environments is a widespread and common process called methanogenesis. The biogenic generation of methane from the molecules of kerogen is achieved by a symbiotic consortium of microorganisms. Syntrophic bacteria of the consortium break down the organic molecules through anaerobic respiration and fermentation into simple, water-soluble compounds (e.g. acetate, CO₂, H₂), which are ultimately transformed into CH₄ by methanogenic archaea.

The method comprises stimulation of nature microbial populations at the margins of black shale and coal bed deposits where the organic matter is less mature and hydrologic flows there through are active to produce methane by delivering supplemental nutrients (a treatment referred to as “biostimulation”) with sulfurous acid. Methane production is thus stimulated by delivering water, acid, sulfites, sulfates, and other nutrients to the microbial consortia under anaerobic conditions to stimulate the syntrophic bacteria and methanogenic archae.

Under anaerobic reducing conditions,

a) denitrification occurs: CₐH_{b}O_{c}+(4a+b/4−c/2)O₂→aCO₂+(2b −2a+c) H2O+(4a+b−2c) OH⁻+(2a+1/2b−c) N₂

b) sulfate reduction occurs: CₐH_{b}O_{c}+(2/5a+1/10B−1/5c)SO₄→aCO₂+(2/5b−2/5a+1/5c) H₂O+(2/5a+1/10b−1/5c) H2S

c) methanogenisis occurs: CₐH_{b}O_{c}+(a−b/4−c/2)H₂O→(a/2−b/8+c/4)CO₂+(a/2+b/8−c/4)CH₄ (Buswell reaction)

The Buswell reaction results from three separate biological reactions by three different types of syntrophic microorganisms:

a) acetogenic bacteria generate acetate and hydrogen that is toxic to themselves:

CₐH_{b}O_{c}+(a−c)H₂O→+1/2aCH₃CO⁻₂+1/2aH⁺½(b−2c)H₂

b) hydrogenotrophic methanogens remove the hydrogen to protect the acetogenic bacteria:

CO₂+4H₂→CH₄+2H₂O

c) acetoclastic bacteria use the acetate to form methane and carbon dioxide:

CₐH_{b}O_{c}+H⁺→CH₄+CO₂

As anaerobic conditions are generally required for the microbial consortia, sulfur dioxide (SO₂) is injected into water to be injected into the kerogen beds forming a weak acid to provide H⁺, SO₂, SO₃⁼, HSO₃⁻, dithionous acid (H₂S₂O₄), and other sulfur intermediate reduction products. Sulfur dioxide acts as a strong reducing agent in water and in the presence of minimal oxygen no additional acid is required to be added to insure the electrical conductivity level of the sulfur dioxide treated water is sufficient for release of electrons from the sulfur dioxide, sulfites, bisulfites, and dithionous acid to form a reducing solution. The sulfur dioxide treated water provides the oxidation/reduction potential within the black shale and coal bed for the syntrophic bacteria of the consortium break down the organic molecules through anaerobic respiration and fermentation into simple, water-soluble compounds (e.g. acetate, CO₂, H₂), which are ultimately transformed into CH₄ by methanogenic archaea.

The acetoclastic bacteria chemical reaction is also driven to the right to form more methane by the addition of the weak sulfurous acid:

CH₃CO⁻₂+H⁺→CH₄+CO₂.

The oxidation/reduction potential of the sulfurous acid in milivolts for anoxic conditions with no dissolved oxygen is usually between +50 and −100 mV, although the exact potential is dependent upon the consortium bacteria present.

The sulfurous acid also acts to dissolve and free up carbonates/bicarbonates to open up pores and channels in the black shale and coal beds to better deliver nutrients and carbon dioxide to the microbial consortia. Sulfurous acid is a powerful reducing agent, which removes oxygen; thereby insuring anaerobic conditions for the syntrophic bacteria and methanogenic archae. The freed up added CO₂ also drives to the right the chemoautotrophic assimilation of CO₂ by the hydrogen consuming methanogens to produce more methane:

CO₂+4H₂→CH₄+2H₂O

If sufficient microbial consortia are not present in the kerogen beds, cultures of syntrophic bacteria and methanogenic archae may be delivered along with the sulfurous acid into the black shale and coal beds to start the methanogenesis process.

Generally, the source-rocks of interest are the Lower Jurassic black shales of the eastern Paris Basin (i.e. type II kerogens). Corings into various points within the shales are drilled to deliver the sulfurous acid at various points within the hydrologic flows of the bed. Other drill holes penetrate the bed at various points to collect the generated gases.

The presence of methane in sample culture extracts of the sulfurous acid correlates with the detection of archaea and methanogens by qPCR. Thus it may be necessary to monitor the presence of methanogens in the sulfurous acid microcosms by periodic sampling.

If other bacterial, archaeal and methanogen populations are involved in the production of methane or petroleum, the oxidation/reduction potential of the sulfurous acid solutions may be modified to stimulate these other bacterial, archaeal and methanogen populations. For example, in the unlikely event that aerobic conditions are alternatively required, oxygen and additional acid may be injected into the sulfur dioxide (SO₂) water to provide H⁺, SO₂, SO₃⁼, HSO₃⁻, dithionous acid (H₂S₂O₄), and other sulfur intermediate reduction products forming a sulfur dioxide treated water to insure that the electrical conductivity level of the sulfur dioxide treated water is sufficient to accept electrons to create an oxidizing solution. The oxidation/reduction potential in millivolts for oxidizing is between −50 to −150 mV under aerated conditions with sufficient free oxygen, alkalinity, pH, temperature and time.

The production of methane via biostimulation may thus be used with sub bituminous coal beds to generate gas from immature source-rocks as well as shale deposits.

To generate any microbial gas play, the hydrologic framework is critical for the natural inoculation of the microorganisms. Basin margins, where the organic matter is less mature and fractures therein more open, should be targeted to allow nutrients to penetrate the deposit.

The foregoing method employing sulfurous acid to deliver bacterial, archae and methanogen populations with nutrients under anaerobic conditions for biostimulation produces methane and petroleum from kerogen and sub bituminous coal beds are a faster rate than that produced by geophysical production.

### DESCRIPTION OF THE DRAWINGS

FIG. 1 is a drawing of the synthetic carbon cycle.

### DESCRIPTION OF THE ILLUSTRATED EMBODIMENTS

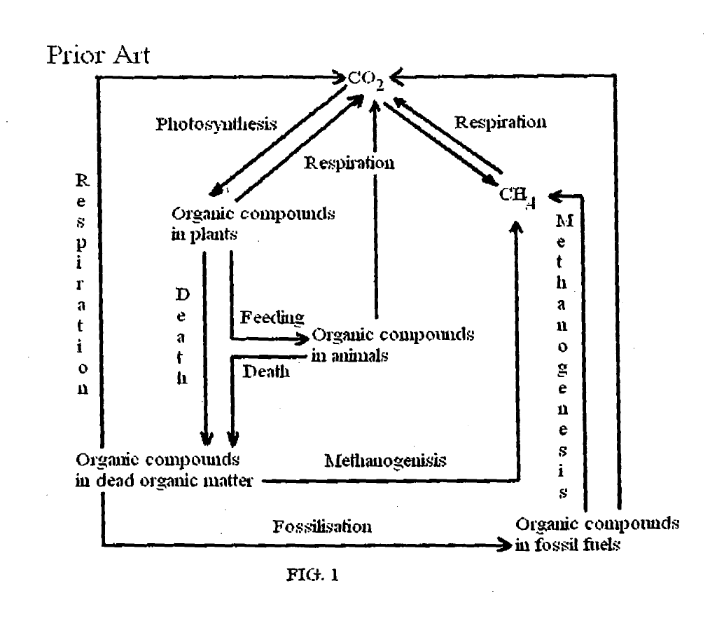

FIG. 1 is a drawing of the synthetic carbon cycle produced by Haeseler & Behar, in their article “Methanogenisis: A Part of the Carbon Cycle with Implication for Unconventional Biogenic Gas Resources” presented at the Natural Gas Geochemistry: Recent Developments, Applications and Technologies seminar May 9-12, 2011 at the AAPG HEDBERG Conference in Beijing, China, which illustrates methanogenisis of the present method acting on organic compounds in fossil fuels to produce methane and hydrocarbon compounds. The present method delivers water, sulfur nutrients, and carbonates to fossil beds under anaerobic conditions for biostimulation of the symbiotic consortium of microorganisms to break down organic molecules through anaerobic respiration and fermentation into simple, water-soluble compounds to produce methane and petroleum from the margins of kerogen and sub bituminous coal beds at a faster rate than that produced by geophysical production.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A biostimulation method of natural microbial populations active at margins of black shale and coal bed deposits where the organic matter is less mature and has hydrologic flows there through comprising:
a. injecting sulfur dioxide into water producing H⁺, SO₂, SO₃⁼, HSO₃⁻, dithionous acid (H₂S₂O₄), and other sulfur intermediate reduction products in sulfurous acid, and
b. applying the sulfurous acid to the black shale and coal bed deposits at a pH sufficient to
i. reduce bicarbonate and carbonate buildup producing CO₂ driving the production of methane by chemoautotrophic assimilation of CO₂ by hydrogen consuming methanogens,
ii. increase porosity and flows through the black shale and coal bed deposits, and
iii. provide SO₂, SO₃⁼, HSO₃⁻, and dithionous acid (H₂S₂O₄) and other sulfur intermediate reduction products to the soluble bicarbonates and carbonate nutrients at an oxidation reduction potential conducive to the growth of microbial consortia under anaerobic conditions to stimulate syntrophic bacteria and methanogenic archaea to produce methane under anaerobic conditions.

2. The biostimulation method according to claim 1, further comprising adding oxygen and additional acid into the sulfurous acid to adjust the oxidation reduction potential is between +50 and −100 mV under aerobic conditions.

3. The biostimulation method according to claim 1, further comprising adding supplemental nutrients to the sulfurous acid.

4. The biostimulation method according to claim 1, further comprising adding syntrophic bacteria and methanogenic archaea to the sulfurous acid to inoculate the black shale and coal bed deposits.
